(19) United States
(12) Patent Application Publication
(10) Pub. No.: US 2013/0230494 A1
(43) Pub. Date: Sep. 5, 2013

(54) Cellular Preparations For Wound Management

(71) Applicant: Marcia Simon, Stony Brook, NY (US)

(72) Inventor: Marcia Simon, Stony Brook, NY (US)

(73) Assignee: The Research Foundation of State University of New York, Albany, NY (US)

(21) Appl. No.: 13/699,457

(22) PCT Filed: May 25, 2011
(86) PCT No.: PCT/US11/00934

§ 371 Date: May 13, 2013

Related U.S. Application Data
(60) Provisional application No. 61/348,144, filed on May 25, 2010.

(51) Int. Cl. C12N 5/071; A61K 45/06; C12N 15/85; A61K 35/36

(52) U.S. Cl. CPC C12N 5/0625; A61K 35/36; A61K 45/06; C12N 15/85; C12N 5/0629
USPC 424/93.7; 435/374

(57) ABSTRACT

Disclosed herein are methods of preserving or preparing cell-based compositions for use in wound management. The methods can be carried out by steps including: (a) providing skin cells; (b) treating the skin cells with a monosaccharide; (c) treating the skin cells with a disaccharide; and (d) lyophilizing the skin cells.

### CROSS-REFERENCE TO RELATED APPLICATIONS

This application claims the benefit of the filing date of U.S. Application No. 61/348,144, filed May 25, 2010. For the purpose of any U.S. application that may issue from the present application or from an application claiming the benefit of U.S. Application No. 61/348,144, the entire content of that earlier-filed application is hereby incorporated by reference herein.

### STATEMENT REGARDING FEDERALLY FUNDED RESEARCH

This invention was made with government support awarded by the United States Army (USAMRAA) under Grant No. W81XWH0820013. The U.S. government has certain rights in this invention.

### FIELD OF THE INVENTION

The present invention relates to cellular preparations for wound management and, more particularly, to freeze-dried preparations including cells such as keratinocytes.

### BACKGROUND

The skin is one of the organs most subject to injury, and repair is a complex process that involves inflammation, formation of granulation tissue, epithelialization, and remodeling of the connective tissue matrix. Healing is best when there is a restoration of the skin, including the dermis and epidermis, in such a way that the resulting scar tissue closely resembles unwounded skin in its structure and function.

The upper part of human skin is composed of the epidermis, which contains mostly keratinocytes or epithelial cells, but also houses other cells types including melanocytes, Langerhans cells, and Merkel cells. The epidermis is stratified, reflecting the state of keratinization. The dermis is composed of connective tissue, including fibroblasts, other connective tissue cells, and connective tissue matrix substances. Blood vessels, nerves, sensory organs, sweat glands, sebaceous glands, and hair follicles are also present in the dermis, and proliferating keratinocytes in the stratum basal are attached to the dermis via the basement membrane.

Clinical and animal experiments have demonstrated healing of acute wounds (eg burns) with cultured keratinocytes applied, for instance as sheets. Applying cultured keratinocyte grafts may also suppress hypertrophic scar formation and keloid formation. Initially, autografts were used, which were prepared by growing keratinocytes isolated from a patient's own skin. Confluent, differentiated keratinocytes were then detached from the culture dish and applied as a sheet, with basal cells facing downwards on the wound. About 3 weeks are needed before a reasonable amount of keratinocytes can be cultured for application as an autograft. Even then, the amount may not be sufficient to cover the entire wound surface. Allografts have also been studied and used. These grafts do not provide a permanent skin replacement and are replaced by the host's (patient's) own skin cells. However, these grafts may promote wound healing when used on acute wounds (e.g. partial thickness burn wounds) and chronic wounds which encompass ulcers both of which may be treated with meshed split skin autografts. Cells used for the preparation of allografts must be checked for multiple pathogens such as HIV or HBV to prevent potential disease transmission.

### SUMMARY OF THE INVENTION

The present invention is based, in part, on our studies of freeze-dried preparations that are made from skin cells and can be used to treat a wide variety of wounded tissues. The preparation methods can be carried out with any type of cell normally found within the skin, with combinations of those cell types, and with stem cells differentiated into skin cells or precursors thereof. For example, one can: (a) provide skin cells; (b) treat the cells with a monosaccharide (e.g., glucose); (c) treat the cells with a disaccharide (e.g., trehalose); and (d) lyophilize the cells. More generally, the treated cells can be dried or lyophilized with a conventional drying or desiccation technique or lyophilized by freeze-drying. While the methods are not so limited, there may be advantages to rapidly freezing and lyophilizing the treated cells because this may preserve more of the protein activity within the cell-based preparations. Following lyophilization, the cell-based preparations can be rehydrated and centrifuged. The supernatant can then be removed and used in accordance with the treatment methods described below for the cell-based preparations.

The cells can be obtained from the epidermis (or, if desired, a stratum thereof), the dermis, or both layers of the skin or from the epithelia and lamina propria of the oral mucosa. More particularly, the cells can be fibroblasts, keratinocytes, Merkel cells, melanocytes, Langerhans cells, stem cells (e.g., stem cells differentiating or differentiated into skin cells), mesenchymal cells, or any combination thereof. The cells can be obtained from a variety of sources, and the source of the cells may coincide with the patient to be treated. For example, the preparations can be made from human cells to treat human patients or from other mammals to treat, respectively, the mammal to which they are later administered. Thus, the present methods can be used to produce cells to treat humans and for veterinary use.

Treating the cells may be achieved by exposing the cells to the monosaccharide and subsequently to the disaccharide (e.g., in a solution that may or may not include additional agents). The monosaccharide can be glucose (or dextrose, the D-isomer of glucose), fructose, furanose, galactose, mannose, pyranose, ribose, xylose, or a combination thereof. The disaccharide can be cellobiose, lactose, lactulose, maltose, sucrose, trehalose, or a combination thereof. The cells can be exposed to the monosaccharide for at least or about 5 minutes (e.g., 5-60 minutes (e.g., at least or about 5, 10, 15, 20, 25, 30, 45, or 60 minutes), optionally washed, and then exposed to the disaccharide for at least or about 5 minutes (e.g., 5-60 minutes (e.g., at least or about 5, 10, 15, 20, 25, 30, 45, or 60 minutes). The concentrations of the monosaccharide(s) and disaccharide(s) can also vary. For example, the cells can be exposed to a monosaccharide present in a solution at about 0.1-3.0 M (e.g., at least or about 0.1, 0.2, 0.3, 0.4, 0.5, 0.6, 0.7, 1.0, 1.2, 1.5, 1.6, 1.67, 1.7, 1.73, or 1.8 M and ranges therebetween (e.g., 1.5-1.8 M). In our studies, we have worked with DMEM supplemented with 1.67 M glucose, and that medium, supplemented as noted, can be used in the present methods (with glucose, another monosaccharide, or a combinations thereof). The cells can be exposed to the disaccharide present in a solution at at least or about 0.1-2.5 M (e.g., about 0.2, 0.3, 0.4, 0.5, 0.6, 0.7, 1.0, 1.5, 2.0, or 2.5 M and ranges therebetween (e.g., 0.2-2.0 M). In our studies, we have worked with DMEM supplemented with about 0.23 M trehalose, and that medium, supplemented as noted, can be used in the present methods (with trehalose, another disaccharide, or a combination of disaccharides).

Alternatively, or in addition, the cells can be exposed to a poloxamer. For example, the methods of making the preparations of the invention can be carried out by exposing the cells to a poloxamer and then exposing them to a disaccharide such as trehalose or another alpha-linked disaccharide. Alternatively, the poloxamer can be included with the solution comprising a monosaccharide and/or the solution comprising the disaccharide to which the cells are exposed and thereby treated. As is understood in the art, poloxamers are nonionic triblock copolymers composed of a central hydrophobic chain of polyoxypropylene (poly(propylene oxide)) flanked by two hydrophilic chains of polyoxyethylene (poly(ethylene oxide)).

Alternatively, or in addition, the cells can be exposed to a large protein such as serum albumin. Thus, in another embodiment, the methods of making the preparations of the invention can include the step of exposing the cells to serum albumin (e.g., to a solution containing serum albumin; a solution containing a monosaccharide and serum albumin; a solution containing a disaccharide and serum albumin; or a solution containing a poloxamer and serum albumin). The serum albumin can be obtained from a variety of sources, including a human or other mammal, and the amount present (e.g., in a buffer used prior to or in the freeze-drying process) can vary from about 1-10 mg/ml (e.g., about 5 mg/ml).

Upon freeze drying and, optionally, rehydration and further processing (e.g., centrifugation or a like process that separates particulate material from supernatant), the cells and cell-based preparations (e.g., supernatant) can be formulated in various ways for application to the skin or other wounded tissue. For example, the cells or cell products can be formulated as a dry powder, suspension, or solution. In other embodiments, the cells or cell products can be formulated as a gel, cream, ointment, or biocompatible matrix. As noted and described further below, cells treated as described herein (for example, with a monosaccharide and disaccharide and/or with a poloxamer and/or serum albumin), can be harvested and later processed to remove cellular debris (e.g., cell membranes such as the plasma membrane and/or nuclear components). For example, the treated cells can be centrifuged prior to use. Thus, upon rehydration and centrifugation, the present methods can generate compositions that include cell fragments rather than intact cells. We may therefore refer to the compositions of the present invention as “cell-based” or as a “cell (or cellular) product.”

More specifically, centrifugation (or a like technique that collects, separates, concentrates, or pellets cells (e.g., filtration)) can be incorporated as a step in the present methods as follows. One would prepare or preserve a cell-based composition by providing a cell (e.g., a skin cell) that has been treated as described herein (e.g., by exposure to a monosaccharide and disaccharide and/or exposure to a poloxamer and/or serum albumin); lyophilize cellular product; and rehydrate the lyophilized cellular product. The rehydrated cellular product can be used directly in wound treatment; manipulated further (e.g., by incorporation in a delivery device); or centrifuged (or filtered) prior to use (e.g., prior to use in wound treatment or prior to further manipulation).

The cells employed in the present methods can be genetically engineered. For example, the cells can be transduced to express or overexpress a growth factor (e.g., an epidermal growth factor, fibroblast growth factor, or biologically active fragments thereof). Similarly, the cells can be genetically engineered to exhibit resistance to activated T cell-mediated cytotoxicity and/or to reduce immunogenicity. For example, immunogenicity can be reduced by inhibiting the expression of MHC I or expressing vIL-10 (viral interleukin-10) alone or in combination. Alternatively, or in addition, one can inhibit one or more of the cytokines that enhance inflammation, as excessive inflammation can impede wound healing. Alternatively or in addition, the preparations can include agents that reduce inflammation or the stress response. For example, the lyophilization buffer and/or a rehydration medium can include an agent that limits reactive oxygen species (e.g., an anti-oxidant or anti-inflammatory agent).

In another aspect, the invention features preparations of cells made by the methods described herein and kits that include such cells and instructions for use.

While the invention is not limited to compositions that promote wound healing by any particular mechanism, cells treated as described herein may promote keratinocyte migration better than a comparable preparation (e.g., a comparable freeze-dried preparation) of cells that was not exposed to a monosaccharide and a disaccharide. This can be assessed in a scratch assay or Boyden chamber assay of trypsinized cells. Cells treated as described above may promote fibroblast migration better than a comparable but untreated preparations of cells. This can be assessed in an assay that does not require production of cell suspensions to test migration. Rather cells may be grown in adjacent chambers which are removed to allow observation of cell migration between the two cell containing areas; this may be accomplished using an Ibidi chamber. Alternatively, movement of cells out of a matrix droplet and onto or into another surface or matrix may be used as an assay.

In another aspect, the present invention features methods of promoting wound healing in a patient by administering to a wound, on the patient's skin, a therapeutically effective amount of a preparation of cells or cell product as described herein. The patient can be a human patient, and the wound may be one that is sustained by intentional or unintentional trauma. For example, the wound may have been sustained in the course of a surgical procedure or in the course of a fire, altercation, motor vehicle accident, or sporting event. The wound can be a cut, burn, or ulceration, and may be associated with a disease process (e.g., a diabetic ulcer). The wound can be a partial thickness wound. While we have emphasized wounds to the skin, the invention is not so limited. The present methods can be applied to treat other injuries, such as those to the eye, mouth, or other mucous membranes.

As the present compositions exhibit stability, particularly in dried or powdered form, and are relatively inexpensive to manufacture, we envision creating a bank of cell-based preparations (derived, for example, from allogeneic keratinocytes) for treatment of thousands and possibly tens of thousands of individuals. We also expect the present compositions to be stable at or around ambient temperature, reducing the cost of storage and shipping and increasing the ease of use in remote areas or over wide areas. The present preparations can remain stable (i.e., useful in wound management) for at least 14 days at room temperature and we expect prolonged stability at cooler temperatures. Lyophilized keratinocytes maintained keratinocyte chemotactic activity for at least 40 days when stored at −80° C. to 50° C., although product stored at 50° C. for 37 days showed reduced ability to promote epithelial sheet migration. The lyophilized keratinocyte product also maintained fibroblast chemotactic activity for at least 35 days when stored at −80° C. to 50° C., although the activity at 35 days was variable. While the invention is not limited to compositions that exert their positive effects on wound healing by any particular mechanism, our work to date suggests that the present compositions can promote keratinocyte migration and promote fibroblast proliferation and migration.

Unless a contrary meaning is clear from the context in which the term is used, we may use the terms “composition(s)” and “preparation(s)” interchangeably.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is a representation of cells treated as described in Example 1 and examined in an epithelial migration or “scratch” assay. The upper photograph shows the lack of migration when cells were exposed to media alone, and the lower photographs shows increased migration promoted by cells treated with glucose and trehalose (or by factors secreted by those cells).

FIG. 2 is a Table of composite data collected from the experiments described in Example 1.

### DETAILED DESCRIPTION

An object of the present invention is to provide compositions and methods for the treatment of cutaneous injuries, including sulfur mustard (bis-(2-chloroethyl) sulfide; HD) injuries, and to mitigate the chronic effects of such injuries. The therapeutic methods can be carried out by administering compositions that include cells that have been lyophilized or desiccated (or fractions thereof, such as non-membranous fractions, which we may also refer to herein as a cell-based preparation or a cell/cellular product). Such cell-based preparations and methods of making them are within the scope of the present invention. We postulated that, because cell-based therapies provide multiple growth factors that either directly or indirectly enhance the expansion and movement of epithelial cells and mesenchymal cells, that such preparations could also provide therapeutic benefit for a wider range of cutaneous injuries (e.g., HD injuries) than do non-biologic occlusive or semi-occlusive dressings.

Cellular Materials:

The present compositions can be made with cells derived from a wide variety of sources, including mammalian and non-mammalian animals; mammalian and non-mammalian cells (e.g., cultured cells); and mammalian and non-mammalian cell lines. For example, the methods can include a step of providing skin obtained from a mammal. For example, skin cells can be obtained from the skin of a pig or other farm or domesticated animal such as a goat, sheep, cow, horse, cat, or dog. The skin cells can also be obtained from a rodent, non-human primate, or human (e.g., from a punch biopsy or excised foreskin). Where the source of the skin cells is bilayered skin, the epidermis and dermis can be separated from one other (e.g., by an enzymatic treatment (with, for example, dispase II), and the methods of the invention can proceed with one or more cell types isolated from the epidermis, the dermis, or a combination of these two layers.

To isolate skin cells (e.g., epidermal keratinocytes) for further processing, the epidermis can be treated with an enzyme such as trypsin, and the resulting single cell suspensions can be grown in culture as described herein or according to methods known in the art. If desired, the cells (e.g., keratinocytes) can be grown on a substrate such as irradiated fibroblasts (e.g., 3T3 cells). The substrate (e.g., a fibroblast layer) can be grown in accordance with methods known in the art and may be grown from the dermis either with or without prior enzymatic disaggregation with enzymes such as collagenase and trypsin. Our studies to date indicate that keratinocytes benefit from feeder cells, as keratinocyte colonies remain small (<2 mm) even after two weeks of incubation with feeding every other day in their absence. Although 3T3 cells are suitable feeders, other cell types such as lethally irradiated fibroblasts and keratinocytes can also be used. Human keratinocyte colonies stained with rhoadamine are readily distinguishable from feeder cells, including 3T3 cells, human fibroblasts, and human keratinocytes. Cells useful in the present methods can be obtained from males or females, and if desired in assessing cell cultures (e.g., keratinocyte cultures on a fibroblast feeder layer), one can distinguish male cells (e.g., male keratinocytes) from female cells (e.g., female fibroblasts). For example, male cells can be visualized by in situ PCR with primers specific to the Y chromosome (see Kawaraski et al., 1995).

Particular cell types that can be isolated from the skin and used in the present methods include fibroblasts, keratinocytes, Merkel cells, melanocytes, and Langerhans cells. These cells can be used in various combinations. For example, the methods can be carried out using keratinocytes alone; keratinocytes and fibroblasts; keratinocytes and Merkel cells, melanocytes, and/or Langerhans cells. One or more of these cell types may be excluded from use in making the present compositions as well.

Where the cells subjected to the present methods are provided from a cell culture, the cells can be cultures of one or more cell types from the dermis, the epidermis, or a combination thereof. For example, methods of making the present cell-based compositions can include the step of providing cells from a culture of keratinocytes (e.g., human or other mammalian keratinocytes), with or without other cell types (e.g., with or without one or more of the other cell types normally found in the skin, as listed above)). Such cultures can be obtained by methods known in the art, and particular culture methods and conditions are described further herein. For example, one can seed keratinocyte cells on a support at a density as low as about 1×10⁴ cells/cm² (e.g., at about 3 to 5×10⁴ cells/cm²) in a culture medium. Commercially available media, such as DMEM and F12, can be used and can be used in combination. For example, one can culture skin cells in DMEM and F12 at a ratio of about 3:1. Other media can be used as well. For example, the medium can be Medium 199, with one or more of the following additives: serum, epidermal growth factor (EGF), hydrocortisone and/or cholera toxin, and possibly insulin, free from non-autologous fibroblasts, and free from organ extracts, particularly pituitary extracts. The cells can then be grown at a conventional temperature (e.g., about 37° C. in a water-saturated atmosphere containing CO₂, preferably in the range of about 1% to about 10%, more particularly in the range of about 2% to about 8%.

Immortalized Cells:

Immortalized cells, including keratinocytes that have undergone spontaneous immortalization, can be obtained from mass culture or single cell clones of cultured cells (e.g., keratinocytes or other skin cell types) grown through multiple passages (generally more than 10). Often the cell lines so derived have an chromosomal aberration involving chromosome 8; typically iso8q. In our studies, we evaluated 23 clones from 4 individuals. Thirteen of these had iso8q chromosomes, and in three clones, the iso8q was the only aberration detected by chromosome spreads. Such cells have been previously described (Rice et al., Mol. Biol. Cell 4(2):185-194, 1993; and Allen-Haffmann, J. Invest. Dermatol. 114(3):444-455, 2000). The cells we have isolated do not appear transformed as measured by their inability to grow in soft agar, maintained growth factor requirements, expression of lecithin retinol acyl transferase, etc. (the experiments utilized cells at passages >40). These cells can be employed in the methods described herein, and compositions comprising cell products from immortalized skin cells are within the scope of the present invention. A potential advantage of immortalized cells is that they may be of use in generating large cell banks without the requirement for new donors.

Differentiating/Differentiated Stem Cells:

Another source of cells for the present methods is stem cells that are differentiating into (or have differentiated into) one or more types of skin cells. The stem cells may be embryonic or adult stem cells, and they may have been originally isolated from a variety of tissue types (e.g., muscle, adipose tissue, bone marrow, or skin). The stem cells may be mesenchymal stem cells.

Genetically Modified Cells:

Any of the cells (e.g., skin cells) used in the present methods can be genetically modified to express a detectable label and/or an active protein. For example, one can transduce keratinocytes (or other cell types) with retroviral vectors comprising genes encoding proteins that promote resistance to various drugs (e.g., puromycin, neomycin, ampicillin, and the like) and genes encoding fluorescent proteins (e.g., GFP, rhodamine, fluorescein, and the like) or growth factors (e.g., EGF, FGF, or biologically active fragments or other variants thereof). Transductants can be selected, for example, through two passages. We observed good expression of green fluorescent protein through human keratinocyte colonies and pig keratinocyte colonies. As noted, genetically tagged cells are useful in in vitro and in vivo wound healing assessments.

Culture Conditions:

Prior to treatment with one or more monosaccharides and one or more disaccharides, the cell types described above can be cultured according to standard tissue culture methods and with media known in the art. For example, the cell cultures can be conducted aseptically at 37° C. in CO₂ incubators. The cells (e.g., keratinocytes) can be grown with a feeder cell layer comprising fibroblasts (e.g., lethally-irradiated Swiss 3T3-J2 cells; Rheinwald and Green, 1975) in modified medium previously described (Randolph and Simon, 1993). More specifically, 3T3-J2 cells can be grown in DMEM containing 10% bovine calf serum (HyClone, Utah) at 37° C. in 7.5% CO₂ and passaged at 70-80% confluence. For use as feeder cells, the cultures can be allowed to reach confluence before being washed twice with Ca, Mg-free isotonic phosphate buffer (pH 7.2; PBS) and incubated with the sodium salt of 0.02% ethylenediamine tetraacetic acid (EDTA), pH 7.2 in PBS. The resultant cell suspension can then be lethally irradiated (6000R). If not used immediately, such feeder layers can be stored at 4° C. for use within about 4 days. Keratinocyte cultures can be prepared in T175 flasks (Costar or NUNC brand obtained from Fisher Scientific) using keratinocytes (0.5×10⁶) and irradiated 3T3-J2 cells (3−4×10⁶) grown in a 3:1 volume:volume solution of DMEM:F12 (Invitorgen, Carlsbad, Calif.) supplemented with 10 ng/ml human recombinant EGF (Austral, San Ramon, Calif.), 5 μg/ml human recombinant insulin (EMD BioSciences, San Diego, Calif.), 0.4 μg/ml hydrocortisone (Sigma Chemical Company, St. Louis, Mo.), 10⁻¹⁰ M cholera toxin (ICN Biochemicals, Costa Mesa, Calif.) and 1.8×10⁻⁴ M adenine (ICN). Accordingly, skin cells useful in the present methods can be grown in medium containing one or more of an EGF, insulin, hydrocortisone; and a nucleotide such as adenine. For passage and preparation of cell suspensions (e.g., keratinocyte suspensions), cultures nearing confluence (e.g., at 60-70% confluence) can be washed twice with PBS and feeder cells (e.g., 3T3-J2 cells) can be removed using, for example, an EDTA (e.g., 0.02% EDTA at pH 7.2 in PBS). Keratinocytes can then be harvested using a 37° C., 5-10 minute incubation with a 1:1 mixture of 0.1% porcine trypsin (ICN Biochemicals, Costa Mesa, Calif.), and 0.01% glucose (Sigma Chemical Company, St. Louis, Mo.) in PBS, pH 7.2-7.4 and 0.02% EDTA in PBS, pH 7.2 (Trypsin/EDTA). Trypsin can be neutralized with 10% fetal bovine serum for cell passage and with soybean trypsin inhibitor for experimentation and cells can be collected by centrifugation for 5 minutes at 500×g. Fibroblasts (e.g., porcine fibroblasts) can be grown in DMEM supplemented with 10% fetal bovine serum (Hyclone, Logan, Utah) at 37° C. and harvested by incubation with Trypsin/EDTA as for keratinocytes.

Treatment with Monosaccharides, Disaccharides, and Analogs Thereof:

The cell types described above, which may be cultured and harvested as described above, can be treated with a monosaccharide and then with a disaccharide. For example, one can prepare a cell suspension that is incubated for about 5-60 minutes (e.g., about 15-30 minutes) with glucose (or dextrose, the D-isomer of glucose), deoxyribose, fructose, furanose, galactose, mannose, pyranose, ribose, xylose, or a combination thereof. The cells can be collected (e.g., harvested) following treatment with the monosaccharide(s) and then incubated for an additional time (e.g., for at least or about 5-60 minutes) with a disaccharide. The disaccharide can include any pairing of the monosaccharides just described and can be cellobiose, lactose, lactulose, maltose, sucrose, trehalose, or a combination thereof. These sugars (and/or analogues or derivatives thereof) can be applied to the cells in buffered media (e.g., DMEM with about 7.0% HEPES as a buffer). Moreover, the sugars (whether monosaccharide or disaccharide) can be included as the D-form, the L-form, or a combination of D- and L-forms. While the cyclic form of sugars predominates and these forms can clearly be used in the present methods, linearized monosaccharides can be used as well.

Derivatives of monosaccharides that can also be employed in the present methods include galactosamine, glucosamine, and 3-O-methyl-D-glucose (e.g., at 0.1-1.0 M).

Further, in some embodiments, the cells can be treated with polysaccharides and, in particular, dextran (a polymer of glucose). For example, the cells (as described herein) can be treated by exposure to a solution containing at least one monosaccharide (e.g., glucose) and at least one polysaccharide (e.g., dextran). For example, the cells can be exposed to a solution comprising 0.1-3.0 M (e.g., 1.0-2.0 M) glucose and about 30-50% (e.g., 40%) dextran (e.g., 4,000-6,000 MW dextran). containing solution and then; e.g., a solution

Prior to drying (e.g., freeze-drying), cells treated as described above with monosaccharides and disaccharides can be placed in a solution supplemented with a lyoprotectant such as glycine, inulin, maltodextrin, polyethylene glycol, polyvinylpyrrolidone, sorbitol, sucrose, trehalose, or any other suitable lyoprotectant or any combination thereof.

Lyophilization:

The preparations can then be dried, and drying may be performed in various ways using techniques known in the art as evaporation, vacuum-drying, spray drying, fluidized bed drying, infrared drying, microwave drying and freeze-drying. The water present in the solution or suspension can be removed in any way that does not negatively impact the properties of the final preparation. For example, in one procedure, any excess medium or other solution in which the cells are contained (e.g., a simple buffer) can be removed (e.g., by centrifugation), before the cells are transferred first to a freezer set at about −20° C. for about 4-5 hours and second to a freezer set at about −80° C. Replicate vials can be lyophilized at 30 mTorr, with lyophilization times optimized. For secondary drying, the temperature can be raised to 20° C. at a rate of about 0.8° C./minute at 50 mTorr.

In another drying or lyophilization process, cells can be incubated either before or after harvest with “loading buffer” comprising 100 mOsm ADSOL, 800 mM trehalose, and 6.6 mM K-phosphate (pH 7.2) for about 15 minutes to about six hours (e.g., 30 minutes to six hours) at 4° C., 23° C., or 37° C. The loading buffer can then be removed by centrifugation and the cells resuspended at ˜10⁷ cells/ml in a buffer (e.g., 100 mOsm ADSOL, 100 mM trehalose, 6.6 mM potassium phosphate (pH 7.2), 15% HES (high molecular weight), and 2.5% human serum albumin). Samples can then be cooled from room temperature (22° C.-24° C.) to about −40° C. with a cooling rate of −1.5° C./minute followed by incubation at −40° C. to −30° C. for about 7 hours at about 30 mTorr. For secondary drying, the shelf temperature can be raised to 20° C. at a rate of 0.8° C./minute at 50 mTorr. Vials will be capped. To provide some guidance as to cell number and volume, which will be generally understood in the art, we have frozen 2×10⁶ cells in 400 μl in 1 cm diameter conical tubes.

Treated cells can also be frozen using either FM1 (DMEM supplemented with 0.28 M glucose, 0.7% HEPES, and 0.49% human serum albumin) or a Ca, Mg-free phosphate buffered saline.

As desired, one can assess various characteristics of the lyophilized cellular preparations, including protein stability (assessed, for example, by an assay for lactate dehydrogenase (LDH) activity) and cell permeability (assessed, for example, by trypan blue exclusion). We have tested various cell-based compositions (e.g., preparations of pig keratinocytes and fibroblasts, rehydrated) for membrane integrity and protein stability, and any of the compositions described herein can be similarly analyzed as needed or desired. These characteristics of the preparations and others (e.g., proliferative potential) can also be assessed upon rehydration (with or without particulate matter reduction) and resuspension.

The lyophilized preparations are within the scope of the present invention as are lyophilized or otherwise dried preparations that have been rehydrated and fractionated (e.g., by centrifugation).

Formulations:

The dried (e.g., freeze-dried or lyophilized) compositions described herein can be frozen, thawed, and/or rehydrated, and compositions in these forms are within the scope of the present invention. Rehydration can be performed at varying temperatures using, for example, both room temperature (˜23° C.) and 37° C. solutions with gentle agitation in, for example, twice the volume of each freeze medium and/or phenol red free-Dulbecco-Vogt Modification of Eagle's Medium (DMEM). As noted, the rehydrated cell-based preparations can be filtered or “spun down” by centrifugation, and the supernatant can be collected. The supernatant and compositions containing the supernatant are within the scope of the present invention and can be applied to wounds or further manipulated (e.g., incorporated in a more complex formulation) for application to a wound site.

Further, due to the inclusion of a pharmaceutically acceptable vehicle, the present compositions can take the form of a gel, a cream, a dry powder, a suspension, a solution, an ointment, or a biocompatible matrix, and additional active agents can be included either by virtue of expression in the cells used as starting materials or by addition to rehydrated preparations. Based on our work to date, it does not appear that the cell-based preparations made as described herein include any significant number of viable cells. As noted above, the cell-based preparations can be centrifuged or similarly treated (e.g., by filtration) to remove particulate material. An advantage of this step lies in the removal of membrane-associated MHC I antigens. Accordingly, cell-based preparations from which some (e.g., <about half), most (e.g., >half) or essentially all (e.g., >95%) of the particulate material has been removed are within the scope of the present invention: Moreover, the process methods of the invention can include a step of removing particulate material (e.g., membranous material) from a rehydrated composition, and the treatment methods of the invention can include a step of administering cell-based preparations with reduced particulates to a wound bed. Our work to date indicates that the supernatant of rehydrated, lyophilized keratinocytes pretreated with 30% glucose and lyophilized with 2% trehalose promotes keratinocyte and fibroblast migration. The efficacy of such compositions may be enhanced by including glucose (0.07-0.15 M) to the lyophilization buffer.

Excipients:

The dried, cell-based preparations described herein can be rehydrated in physiologically acceptable materials that may include a buffering agent. Examples of buffers useful in this regard include citrate buffers, phosphate buffers, carbonate buffers, HEPES, or any other suitable buffer known in the art. Either prior to rehydration or afterward, the preparations can be essentially homogeneous (i.e., free from inconsistencies that render one part of the preparation substantially different from another part of the preparation).

The cells incorporated in the present preparations (e.g., as dried formulations) can be suspended or otherwise contained in an isotonic solution such as sodium chloride or a phosphate buffer.

Other Active and Inactive Agents:

As noted, in some embodiments, the dried preparations can be rehydrated or otherwise formulated (e.g., as a gel, cream, powder, suspension, solution, ointment, or biocompatible matrix) to include growth factors such as EGF and FGF, anti-inflammatory agents, and/or anti-oxidants (e.g., vitamin C or vitamin D).

In certain embodiments, the preparations, when rehydrated, may be formulated to include or exclude one or more antiflocculant and/or antisedimentation agents such as xanthan gum and/or maltodextrin.

In certain embodiments, the present cell-based preparations, either as dried or rehydrated formulations, can be incorporated into a cell delivery system, and such systems are known in the art. For example, the present cell-based preparations can be applied to a wound bed on microcarrier beads or a cellular or acellular matrix (e.g., Alloderm® (LifeCell Corporation, Branchburg, N.Y.); Oasis® (Healthpoint Ltd., Fort Worth, Tex.); Integra® (Dermal Regeneration Sugarland, Tex.); or Transcyte® (Smith and Nephew, La Jolla, Calif.)).

Activity and Stability:

If desired, one can evaluate the ability of post-rehydration preparations to facilitate or enhance wound healing, and these evaluations can be carried out at varying points in time (e.g., 1-10 weeks (e.g., 1, 2, and/or 3 weeks)) after rehydration in order to assess the stability of the preparations (including stability at room temperature). For example, one or more in vitro screening assays can be performed to assess wound healing potential. For example, in vitro analysis of re-epithelialization can be carried out to test a given composition's performance as a feeder layer; in vitro analysis of re-epithelialization and re-vascularization can be carried out to test the migration of keratinocytes, and endothelial cells; in vitro assays for granulation tissue formation can be carried out by testing the promotion of fibroblast migration into a provisional matrix; and in vitro assays of endothelial network formation can be carried out to assess tubulogenesis. These properties can also be assessed in in vivo models, and efficacy can be further tested as a function of both wound closure rate and skin function (e.g., in an existing weanling pig model). We are not suggesting that the present compositions must promote such events to any given extent or degree in order to be useful. However, these analyses can be performed to better understand the present compositions and their properties and capabilities, and one or more analytical steps, such as those listed above, can be carried out as a part of the present methods for preparing and/or assessing cell-based preparations for use as therapeutic agents. Our studies to date indicate that the present compositions remain stable and retain wound-healing capacity weeks (e.g., two weeks or more) after rehydration, even when stored at room temperature.

In the weanling pig model mentioned above, any of the cell-based preparations described herein can be applied to a field of skin of an animal that has be exposed to neat liquid HD to produce superficial dermal injuries within 48 hours. The injured skin can be debrided, and the cell-based preparations can include frozen keratinocytes without fibroblasts; frozen keratinocytes with fibroblasts; lyophilized or desiccated keratinocytes without fibroblasts; and lyophilized or desiccated keratinocytes with fibroblasts. For the sake of clarity, we reiterate that these cell types and combinations of skin cells such as these can be employed in the methods of the invention and cell products derived from these cell types can be found within the cell-based preparations.

To facilitate analysis of the present compositions, one can tag or genetically modify host cells and/or the cells used to make the present compositions with different detectable markers (e.g., different fluorescent proteins) to allow host cells to be distinguished from cells within the preparations. Exposing distinguishable host cells to the preparations of the invention readily allows one to assess the properties and capabilities of the preparations, and preparations generated from cells that were detectably labeled are within the scope of the present invention. Thus, one can make a composition of the present invention by treating detectably labeled (e.g., fluorescently, enzymatically, or radioactively labeled) cells (e.g., dermal and/or epidermal cells) as described herein. Generating the present compositions from detectably labeled cells permits one to more easily analyze the ability of the composition to support clonal growth of keratinocytes; enhance epithelial or endothelial sheet migration; deliver chemoattractants for keratinocytes, fibroblasts and/or endothelial cells; promote transmigration of fibroblasts from fibrin to provisional matrices; and promote endothelial cell tubulogenesis.

In vivo, one can deploy the present compositions in essentially any wound model. For example, analysis in vivo can include an evaluation of biopsies taken from control and treated HD cutaneous wounds. Formalin-fixed tissue can be processed for hematoxylin and eosin staining. Masson's trichrome can highlight dermal collagen, and Movat's pentachrome can highlight elastic fibers. The histopathology of HD-induced lesions has been monitored in animal models (Papirmeister et al., 1991; Petrali et al., 1992; Vogt et al., 1984; and Graham et al., 2006) and in human organ cultures (Moore et al., 1986; Nakamura et al., 1990; Lindsay and Rice, 1996; and Smith et al., 1998). Any of these models and systems can be used in assessing the compositions of the present invention. As appropriate, tissue can also be evaluated with tests such as the histomorphologic scale for rating burn scars developed by Singer et al. (2000). Immunohistochemical analysis can be carried out using commercially available antibodies to localize proteins associated with granulation tissue formation, neovascularization, basement membrane zone remodeling, and re-epithelialization and epidermal differentiation.

Methods of Treatment:

As noted, the compositions described herein can be administered via different carrier systems and drug dosage forms such as creams, ointments, gels, powders, sprays, solutions, suspensions, emulsions, lyophilized powders, and aerosols. The methods of treatment per se can include a step of identifying a patient who would benefit from treatment, and such patients include those with a surface wound caused by, for example, a thermal, chemical, electrical or radiation-induced burn wound of the skin. The treatment can be allogeneic in that the composition applied to the patient can be made from cells provided by a different individual of the same species or autologous in that the composition applied to the patient can be made from that individual's own cells. Patients having burn wounds covered with meshed skin autografts will also benefit from the preparations of the invention, as we expect the preparations will stimulate the closure of the meshed skin interstices. The wounds may constitute full thickness or partial thickness wounds, and mechanical wounds such as incisions, abrasions and lacerations, whether obtained through accident or in the course of a surgical procedure, can also be treated. Other patients amenable to treatment include patients with an ulceration of the skin, such as decubitus, venous and arterial ulcers and ulcers caused by underlying diseases such as diabetes and vasculitis. The injury may also be a corneal wounds or a tympanic membrane lesion. In other embodiments, the patient may have a lesion caused by a pathological condition such as bullous pemphigoid, epidermolysis bullosa, or lupus erythomatosus.

The present compositions can be administered in addition to, or in the context of, other recommended treatment protocols. For example, treatment for individuals exposed to HD currently includes decontamination, fluid management, administration of antipruitics, and deroofing blisters greater than 1 cm in diameter followed by daily cleansing and treatment with topical antibiotics. The current preparations can be administered to the wound site to supplement this protocol. We expect a therapeutic advantage of aggressive debridement of damaged tissue followed by either split-thickness grafting of full-thickness (third degree) injuries or coverage of partial thickness (second degree) injuries by one of several dressings (Graham et al., 2006). For added benefit, it may be necessary to debride the tissue peripheral to the exposure site and thereby remove alkylated basement membrane and sublethally damaged epithelial cells that may either inhibit re-epithelialization or contribute to chronic skin problems sometimes associated with HD injuries. Thus, debridement may be included as a step in treatment methods employing the current preparations.

The properties of the present compositions lend them to ambulatory and battle-area care, and patients suffering from exposure to sulfur mustard (HD) are among those amenable to treatment. HD is an alkylating agent that has been used since the First World War and remains a present day chemical warfare threat; it targets epidermal and mucosal (including ocular and respiratory) surfaces where it causes vessication and impacts immune function. Depending upon exposure, debilitating injuries can develop over several days and can take months to heal, necessitating lengthy hospitalizations and leading to chronic cutaneous and mucosal problems.

### EXAMPLES

### Example 1

We examined the cryoprotective capacity of trehalose on pig keratinocytes and fibroblasts using a 1.67 M glucose pretreatment to enable loading of 0.23 M or 1.46 M trehalose. The procedure entailed preparation of cell suspensions that are pre-incubated for 30 minutes with glucose (1.67 M), harvested by centrifugation and then incubated for an additional 30 minutes with trehalose (0.23 M or 1.46 M). Afterwards, freezing was carried out using the protocol reported by Zhou et al. (Cell Preservation Technol. 6:119-122, 2008).

Pig keratinocytes treated in this manner at either trehalose concentration showed reduction in total LDH activity compared to all other treatments (12-16 vs. 19-300D 490/min/2×10⁶ cells) and significant release of LDH even at room temperature (18-19% vs. 5-6%). CFEs were also low at room temperature in the 1.46 M trehalose group (0.3%±0.6%). Post-freeze CFEs were similar for cells incubated with 0.23 M trehalose (0.17%±0.02%) and 1.46 M trehalose (0.18%±0.08%). Both concentrations of trehalose limited increased membrane permeability measured by trypan blue staining and LDH release due to the freezing process. Neither protectant medium was effective at preventing membrane permeabilization or maintaining CFE subsequent to lyophilization.

The cryoprotective capacity of trehalose was also tested on pig fibroblasts. In contrast to pig keratinocytes, the total LDH activity and CFE of these cells at room temperature was not altered by the treatments. Maintenance of proliferative potential post-freeze was modestly enhanced using 0.23 M trehalose (0.68±0.06% CFE) compared to 1.46 M trahalose (0.49±0.05% CFE). In a single experiment carried out using 0.23 M trehalose without glucose pretreatment, it appeared that glucose pretreatment increased LDH release at room temperature, but prevented LDH release and loss of CFE post-freeze.

Preliminary experiments to promote desiccation tolerance were carried out using cells grown to 70-80% confluence. These cells were harvested with trypsin/EDTA, neutralized with serum and then resuspended (5×10⁶ cells/mL) in keratinocyte medium (Randolph and Simon, J. Biol. Chem. 268:9198-9205, 1993) in a base medium of DMEM 0.5% human serum albumin, and 30 mM HEPES with either 30 mg/ml inulin, 50 mM trehalose or 150 mM trehalose. Cells in keratinocyte medium or inulin-containing medium were pre-incubated for 3 hours at room temperature with rotation prior to desiccation and cells in the trehalose media were incubated at 4° C. for 24 hours, followed by 3 hours at room temperature prior to desiccation. Desiccation was carried out using 20 μL droplets of cells spotted onto P35 plates. After 60 minutes of desiccation, the water content of cells in keratinocyte media and inulin-containing medium was 0.094 g/g dry cells and 0.100 g/g dry cells, respectively (Gordon et al., Cryobiology 43:182-187, 2001; Ma et al., Cryobiology 51:15-28, 2005; Puhlev et al., Cryobiology 42:207-217, 2001).

Keratinocytes resuspended in either keratinocyte medium or inulin-containing medium maintained colony forming cells following desiccation for up to 60 minutes. Keratinocytes resuspended in the trehalose media maintained colony forming cells only in the 45 minute time point. There was significant variability in CFEs within each group. Normalization of CFE to CFE of trypan blue cells revealed that desiccation impacted the colony forming cells similarly to the entire population.

The potential for using XTT hydrolysis as a measure of viability was assessed using Cell Proliferation Kit II (XTT). Assay optimization to ensure linearity with cell number was first carried out. Pig keratinocytes were grown to 80% confluence, harvested using trypsin/EDTA, and resuspended in PBS to 0.25×10⁶, 0.50×10⁶, 1.0×10⁶ and 2.0×10⁶ cells/400 μL. Using a 96-well plate format in an ELISA reader, reactions containing 50 μL of each cell suspension and 25 μL of the XTT solution were incubated at room temperature and formazan product measured as an increase in the absorbance at 490 nm. Assays comprising 0.25-0.50×10⁶ cells were in the linear range. Therefore, subsequent experiments were assayed using 0.50×10⁶ cells. We obtained data from cells exposed to multiple cryoprotectants (5% and 40% dextran 6000, 1.67 M glucose pretreatment of cells incubated with either 0.23 M or 1.46 M trehalose, and 1 M and 2 M glucose). Each of these treatments had been found to support maintenance of LDH activity through freezing, lyophilization and rehydration. Maintenance of CFE was variable post-freeze and was lost consequent to lyophilization and rehydration. No significant correlation was found between the various assays and even though formazan formation was reduced post-freeze in a manner consistent with reduction in CFE, no further reduction was detected post-lyophilization. Thus, residual activity is maintained even after all proliferative capacity has been lost.

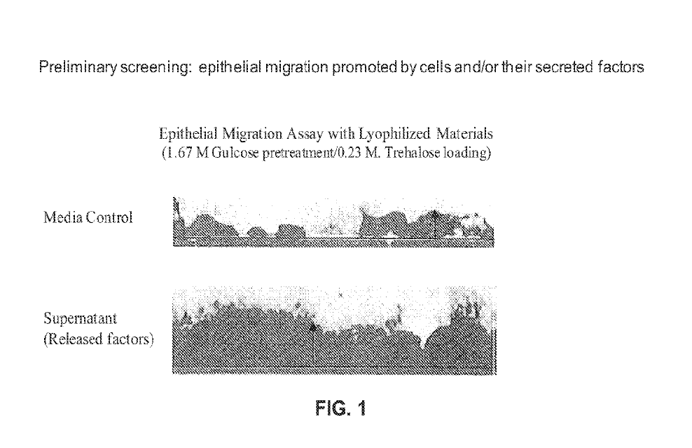

In vitro assays of therapeutic potential of frozen and lyophilized cells and their released factors were monitored using a modified scratch assay. In this screening, the use of trehalose (0.23 M) on cells pretreated with 1.67 M added glucose yielded the most promising results. These samples promoted the greatest epithelial migration when compared to any of the control media tested to date (FIG. 1).

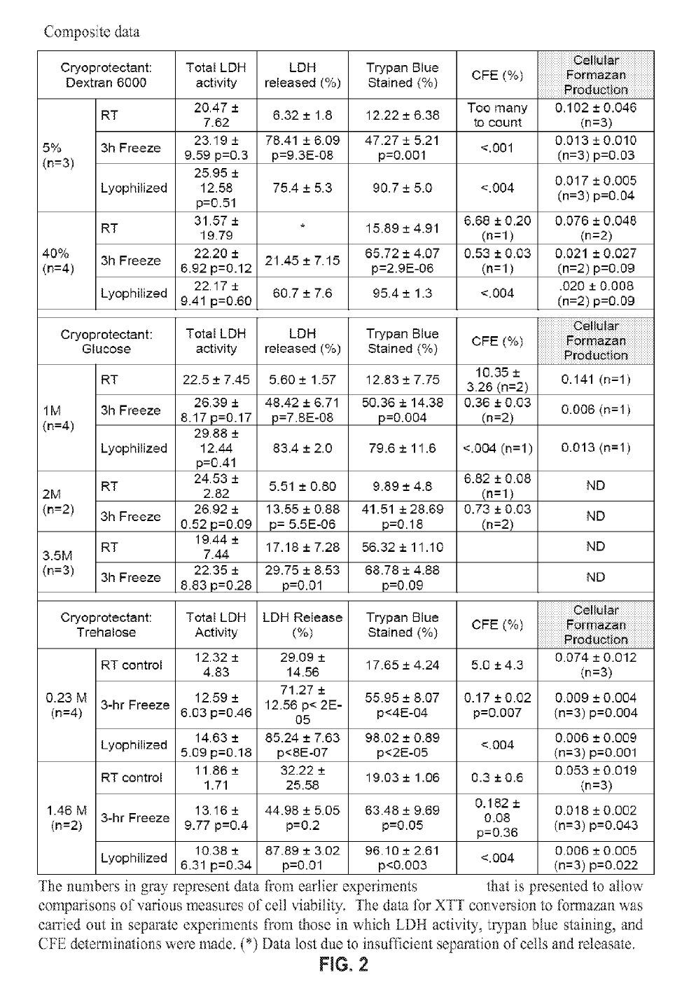

Based on this work, we concluded that using a modified scratch assay, a potential therapeutic benefit for partial thickness HD-wounds was identified using factors derived from cells pretreated with 1.67 M glucose and lyophilized with 0.23 M trehalose. Because multiple conditions result in release of cell factors, this data supports a role for a factor that may be identifiable. The capacity for XTT conversion to formazan does not completely correlate with loss of colony forming capacity, as residual activity is maintained in all lyophilized samples in spite of a CFE<0.004%. We also found that pig fibroblasts are less sensitive than pig keratinocytes to the adverse effects of glucose/trehalose. Finally, the CFE of desiccated keratinocytes when normalized to trypan blue excluding cells is within two-fold of that of non-desiccated samples. This suggests that both proliferative and differentiated cells are similarly affected by the process. This differs from the freezing process in which the proliferative population is more sensitive to the preservation procedure. Composite data obtained from the experiments described above is shown in FIG. 2.

A number of embodiments of the invention have been described. Nevertheless, it will be understood that various modifications may be made without departing from the spirit and scope of the invention. Accordingly, other embodiments are within the scope of the following claims.

What is claimed is:

1. A method of preserving or preparing cell-based compositions for use, the method comprising:
(a) providing skin cells;
(b) treating the skin cells with a monosaccharide;
(c) treating the skin cells with a disaccharide; and
(d) lyophilizing the skin cells.

2. The method of claim 1, wherein the skin cells are epidermal cells, dermal cells, or a combination of epidermal and dermal cells.

3. The method of claim 1, wherein the skin cells are fibroblasts, keratinocytes, Langerhans cells, melanocytes, Merkel cells, melanocytes, or a combination thereof.

4. (canceled)

5. The method of claim 1, wherein treating the cells with a monosaccharide comprises exposing the cells to a solution comprising glucose at about 0.5-2.0 M.

6. The method of claim 5, wherein the solution comprises glucose at about 1.7 M.

7. The method of claim 1, wherein treating the cells with a disaccharide comprises exposing the cells to a solution comprising trehalose at about 0.1-0.5 M.

8. The method of claim 7, wherein the solution comprises trehalose at about 0.23 M.

9. The method of claim 1, further comprising treating the cells with a poloxamer and/or serum albumin.

10. The method of claim 9, wherein the poloxamer and/or the serum albumin is present in a solution comprising the monosaccharide and/or a solution comprising the disaccharide.

11. (canceled)

12. The method of claim 1, further comprising formulating the cells as a dry powder, suspension, solution, gel, cream, ointment, or biocompatible matrix, optionally further comprising a physiologically acceptable excipient and an anti-oxidant or anti-inflammatory agent.

13. The method of claim 1, wherein the cells are genetically engineered.

14. The method of claim 13, wherein the cells are genetically engineered to overexpress a growth factor or underexpress MHC I and/or a cytokine that enhances inflammation.

15. (canceled)

16. The method of claim 1, wherein lyophilizing the cells comprises freeze-drying the cells.

17. The method of claim 1, wherein the cell is a keratinocyte that is not transformed; appears genetically stable; and expresses the gene LRAT.

18. A cell-based preparation made by the method of claim 1.

19. The cell-based preparation of claim 18, wherein the preparation is more stable at room temperature at a given time following the lyophilizing step than is a second preparation that was not treated with a monosaccharide and a disaccharide but is otherwise comparable to the cell-based preparation.

20. A method of promoting wound healing in a patient, the method comprising administering to a wound, on the patient's skin or the surface of the eye, a therapeutically effective amount of the cell-based preparation of claim 18.

21. (canceled)

22. The method of claim 20, wherein the wound was sustained by intentional or unintentional trauma.

23. The method of claim 22, wherein the wound is an intentional wound sustained in the course of a surgical procedure or an unintentional wound sustained in the course of a fire, altercation, motor vehicle accident, sporting event, or combat.

24. The method of claim 22, wherein the wound is a cut, burn, ulceration, or partial thickness wound.

25. (canceled)
